(19) United States
(12) Patent Application Publication
(10) Pub. No.: US 2024/0029249 A1
(43) Pub. Date: Jan. 25, 2024

(54) METHOD AND SYSTEM FOR PROCESSING COMPUTERIZED TOMOGRAPHY IMAGES

(71) Applicant: Sheba Impact Ltd., Ramat-Gan (IL)

(72) Inventors: Yael MARDOR, Hertzelia (IL); David LAST, Jerusalem (IL); Dianne DANIELS ELMAKIAS, Ramat-HaSharon (IL); Shirley SHARABI, Ramat Gan (IL); Jair BAR, Ramat Gan (IL); Nadav FORIE, Ramat Gan (IL); Liran LEVY, GeAlya (IL); Edith Michelle MAROM, Tel Aviv (IL)

(73) Assignee: Sheba Impact Ltd., Ramat-Gan (IL)

(21) Appl. No.: 18/376,460

(22) Filed: Oct. 4, 2023

Related U.S. Application Data
(63) Continuation of application No. PCT/IL2022/050361, filed on Apr. 6, 2022.
(60) Provisional application No. 63/171,131, filed on Apr. 6, 2021.

(51) Int. Cl. G06T 7/00; G06T 7/254; A61B 6/03; A61B 6/00

(52) U.S. Cl. CPC G06T 7/0012; G06T 7/254; A61B 6/032; A61B 6/481; G06T 2207/10081; G06T 2207/30061

(57) ABSTRACT

A method of analyzing CT data comprises receiving data describing a pre-contrast administration CT scan from a body region of the subject, and a post-contrast administration CT scan acquired from the body region at least 1 minute after initiation of administration of a contrast agent to the subject. A subtraction map is formed by subtracting one of the scans from another. The subtraction map is displayed in a manner that identifies leaking of contrast agent out of blood vessels in the region.

### RELATED APPLICATIONS

This application is a Continuation of PCT Patent Application No. PCT/IL2022/050361 having International filing date of Apr. 6, 2022 which claims the benefit of priority under 35 USC § 119(e) of U.S. Provisional Patent Application No. 63/171,131 filed on Apr. 6, 2021. The contents of the above applications are all incorporated by reference as if fully set forth herein in their entirety.

### FIELD AND BACKGROUND OF THE INVENTION

The present invention, in some embodiments thereof, relates to computerized tomography and, more particularly, but not exclusively, to a method and system for processing computerized tomography images.

The present invention, in some embodiments thereof, relates to image processing and, more particularly, but not exclusively, to a method and a system for processing computerized tomography (CT) images.

CT systems are used to obtain non-invasive sectional images of test objects, particularly internal images of human tissue for medical analysis and treatment. Current CT systems position the test object, such as a patient, on a table within a central aperture of a rotating frame, or gantry, which is supported by a stationary frame. The gantry includes an x-ray source and a detector array positioned on opposite sides of the aperture, within an x-y plane of a Cartesian coordinate system (generally referred to as the “imaging plane”), such that both rotate with the gantry around the test object being imaged. At each of several angular positions along the rotational path of the gantry (also referred to as “projections”), the x-ray source emits a fan-shaped collimated beam which passes through the imaging slice of the test object, is attenuated by the test object, and is received by the detector array. Each detector element in the detector array produces a separate electrical signal indicative of the attenuated x-ray beam intensity, the beam projected from the x-ray source to the particular detector element, incident at its sensor surface. The electrical signals from all the detector elements are collated by circuitry within the rotating frame to produce a projection data set at each gantry angle or projection. Each projection data set is referred to as a “view”, and a “scan” is a set of such views from the different gantry angles during one revolution of the x-ray source and detector array. The scan is then processed by a computer in the stationary frame to reconstruct the projection data sets into a CT image of the slice or cross-section of the test object.

CT can be used as an imaging modality in most parts of the body, particularly the lungs, breast and liver. For example, volumetric CT technique has been available to provide virtually contiguous spiral scans that cover the chest in a few seconds. This technique has greatly reduced CT image artifacts caused by unequal respiratory cycles, partial volume, and cardiac motion. Newer models of the helical CT system are capable of performing the scan and image reconstruction simultaneously.

Known in the art are CT techniques known as “CT perfusion” (CTP), in which perfusion maps that provide the physician with blood flow information at a small, typically few centimeters in diameter, region-of-interest suspected of including a tumor mass. CTP includes repeated imaging during and following dynamic administration of contrast agent. CTP typically involves imaging over approximately 45-80 seconds, at 1-5 second intervals.

### SUMMARY OF THE INVENTION

According to an aspect of some embodiments of the present invention there is provided a method of analyzing CT data describing a body region of a subject administered with contrast agent. The method comprises: receiving data describing a pre-contrast administration CT scan from a body region of the subject, and a post-contrast administration CT scan acquired from the body region at least 1 minute after initiation of administration of contrast agent to the subject. The method also comprises forming a subtraction map from the data by subtracting one of the scans from another, and displaying the subtraction map in a manner that identifies leaking of contrast agent out of blood vessels in the region.

According to some embodiments of the invention, the method comprises acquiring the pre-contrast administration CT scan, administering the contrast agent to the subject, and acquiring the post-contrast administration CT scan.

According to some embodiments of the invention, the acquisition is by a CT scanner, and the method comprises idling the CT scanner between the pre- and the post-contrast administration CT scans.

According to some embodiments of the invention, the post-contrast administration CT scan is acquired t seconds after the contrast administration, the t being at least 80 seconds. According to some embodiments of the invention, t is at least 120 seconds. According to some embodiments of the invention, t is at least 240 seconds. According to some embodiments of the invention, t is at least 360 seconds. According to some embodiments of the invention, t is at least 480 seconds.

According to some embodiments of the invention, there is a plurality of post-contrast administration CT scans, and the method comprises forming a subtraction map for each of the plurality of post-contrast administration CT scans, relative to the pre-contrast administration CT scan, thereby forming a plurality of subtraction maps.

According to some embodiments of the invention, a time delay between acquisition times of the pre-contrast administration CT scan and a respective post-contrast administration CT scan is at least 1 minute, for each subtraction map.

According to some embodiments of the invention, the method comprises identifying on the map a region of microvasculature leakiness, and optionally and preferably displaying also this identification of microvasculature leakiness.

According to some embodiments of the invention, the body region is a lung, and the map displays the lung in its entirety. According to some embodiments of the invention the body region comprises two lungs, and the map displays both the lungs in their entirety.

According to some embodiments of the invention, the method comprises identifying on the map a region of pulmonary vessel leakiness, and optionally and preferably displaying also this identification of pulmonary vessel leakiness.

According to some embodiments of the invention, the method comprises treating the subject for the pulmonary vessel leakiness.

According to some embodiments of the invention, the method comprises identifying on the map a region of inflammation, and optionally and preferably displaying also this identification of inflammation.

According to some embodiments of the invention, the method comprises treating the subject for the inflammation.

According to some embodiments of the invention, the method comprises identifying on the map a region of pulmonary fibrosis, and optionally and preferably displaying also this identification of pulmonary fibrosis.

According to some embodiments of the invention, the method comprises treating the subject for the pulmonary fibrosis.

According to some embodiments of the invention, the displayed subtraction map is color coded with at least three distinct colors, wherein a first color represents subtraction values less than a first predetermined threshold, a second color represents subtraction values more than a second predetermined threshold which is higher than the first predetermined threshold, and a third color represents subtraction values between the predetermined thresholds.

According to some embodiments of the invention, the method comprises identifying vessel leakiness at a region on the map that is colored by the third color, and optionally and preferably displaying also this identification of vessel leakiness.

According to an aspect of some embodiments of the present invention, there is provided a computer software product. The computer software product comprises a computer-readable medium in which program instructions are stored, which instructions, when read by a data processor, cause the data processor to receive data describing a pre-contrast administration CT scan from a body region of the subject and a post-contrast administration CT scan acquired from the body region at least 1 minute after administration of contrast agent to the subject, to form a subtraction map from the data by subtracting one of the scans from another, and to display the subtraction map in a manner that identifies leaking of contrast agent out of blood vessels in the region.

According to an aspect of some embodiments of the present invention there is provided a CT system. The CT system comprises: a CT scanner configured for acquiring CT scans; a controller configured for controlling the CT scanner to acquire a pre-contrast administration CT scan from a body region of the subject, idle the CT scanner for at least 1 minute, and acquire a post-contrast administration CT scan from the body region; and an image processor configured to receive data describing the pre- and post-contrast administration CT scans, to form a subtraction map from the data by subtracting one of the scans from another, and to display the subtraction map in a manner that identifies leaking of contrast agent out of blood vessels in the region.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings and images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flowchart diagram of a method suitable for analyzing CT data according to some embodiments of the present invention;

FIGS. 2A-2C are schematic illustrations of a post-contrast administration CT scan (FIG. 2A), a pre-contrast administration CT scan (FIG. 2B), and a subtraction map (FIG. 2C) formed according to some embodiments of the present invention by subtracting the scan of FIG. 2B from the scan of FIG. 2A;

FIGS. 3A-3D show experimental results corresponding to the schematic illustrations of FIGS. 2A-C;

FIGS. 4A-4D show post-contrast administration CT scans (FIGS. 4A and 4C) and subtraction maps (FIGS. 4B and 4D) obtained for a subject with lung cancer, before treatment (FIGS. 4A-4B) and 2 months post treatment by chemo-radiotherapy (FIGS. 4C-4D);

FIGS. 5A-5C show three subtraction maps each obtained by subtracting a pre-contrast administration CT scan from a post-contrast administration CT scan, for three different subjects without significant vessel leakiness;

FIGS. 6A-6C show three subtraction maps each obtained by subtracting a pre-contrast administration CT scan from a post-contrast administration CT scan, for three different subject with significant vessel leakiness;

FIG. 7 is a schematic illustration of a CT system, according to some embodiments of the present invention;

FIGS. 8A-8H show pre-contrast administration CT scans (FIGS. 8A and 8E) and subtraction maps (FIGS. 8B-D and 8F-H) of two patients recovering from Covid-19 virus, calculated by subtracting the respective pre-contrast administration CT scan from each of 3 post-contrast administration CT scans; and

FIGS. 9A-9D show a post-contrast administration CT scan (FIG. 9A) and subtraction maps (FIGS. 9B-9D) of a patient with lung cancer during immune therapy, calculated by subtracting the pre-contrast scan from 3 post-contrast CT scans.

FIGS. 10A-10F show a pre-contrast administration CT scans (FIGS. 10A, 10C, and 10E) and respective subtraction maps (FIGS. 10B, 10D, and 10F) of patients which underwent lung transplantation.

### DESCRIPTION OF SPECIFIC EMBODIMENTS OF THE INVENTION

The present invention, in some embodiments thereof, relates to computerized tomography and, more particularly, but not exclusively, to a method and system for processing computerized tomography images.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

A CT system is an X-ray system used to produce cross-sectional images of organs. CT systems have become a major diagnostic modality in modem medicine and are widely used for many types of exams. During a chest and abdominal scan, for example, a CT system produces a series of cross sectional images which include lung, soft tissues, liver and bone. Radiologists are able to examine these series of cross sectional images to diagnose a disease in one or more of the scanned organs.

Most exams are performed by subjective viewing of the cross-sectional images on an electronic display. This subjective viewing makes use of subjective discrimination of tissue types by density differences. Generally, CT images present the attenuation coefficient of the X-ray radiation. The attenuation coefficient value typically ranges from −1000 Hounsfield Units (HU), which is typical for air, to 2500 HU, which is typical for bones.

The Inventors realized that CT scans can provide information regarding leakiness of blood vessels. The Inventors particularly realized that such information can be obtained by acquiring two or more CT scans of a tissue region. Unlike conventional techniques that utilize multiple CT multiple acquisitions (e.g., CTP), the technique of the present embodiments uses a relatively small number of CT scans, and is therefore advantageous because it exposes the subject to a relatively low radiation dose, which is not the case in conventional CTP. An additional advantage of the technique of the present embodiments is that it is generally model independent, unlike CTP which requires a mathematical model to analyze a multiplicity of CT scans in order to determine the blood flow and volume characteristics.

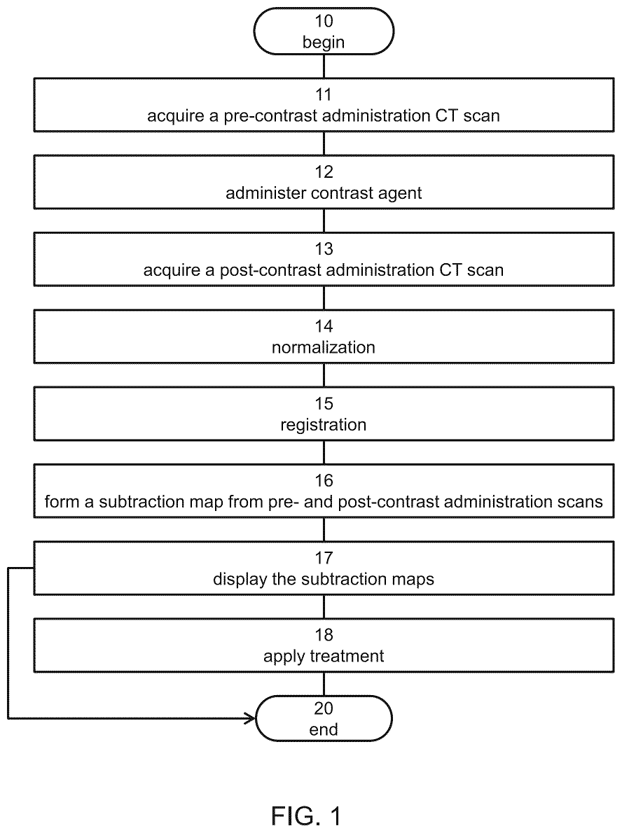

FIG. 1 is a flowchart diagram of a method suitable for analyzing CT data according to various exemplary embodiments of the present invention. It is to be understood that, unless otherwise defined, the operations described herein below can be executed either contemporaneously or sequentially in many combinations or orders of execution. Specifically, the ordering of the flowchart diagrams is not to be considered as limiting. For example, two or more operations, appearing in the following description or in the flowchart diagrams in a particular order, can be executed in a different order (e.g., a reverse order) or substantially contemporaneously. Additionally, several operations described below are optional and may not be executed.

At least part of the operations described herein can be can be implemented by an image processing system, e.g., a dedicated circuitry or a general purpose computer, configured for receiving data and executing the operations described below. At least part of the operations can be implemented by a cloud-computing facility at a remote location.

Computer programs implementing the method of the present embodiments can commonly be distributed to users by a communication network or on a distribution medium such as, but not limited to, a floppy disk, a CD-ROM, a flash memory device and a portable hard drive. From the communication network or distribution medium, the computer programs can be copied to a hard disk or a similar intermediate storage medium. The computer programs can be run by loading the code instructions either from their distribution medium or their intermediate storage medium into the execution memory of the computer, configuring the computer to act in accordance with the method of this invention. During operation, the computer can store in a memory data structures or values obtained by intermediate calculations and pulls these data structures or values for use in subsequent operation. All these operations are well-known to those skilled in the art of computer systems.

Processing operations described herein may be performed by means of processer circuit, such as a DSP, microcontroller, FPGA, ASIC, etc., or any other conventional and/or dedicated computing system.

The method of the present embodiments can be embodied in many forms. For example, it can be embodied in on a tangible medium such as a computer for performing the method operations. It can be embodied on a computer readable medium, comprising computer readable instructions for carrying out the method operations. In can also be embodied in electronic device having digital computer capabilities arranged to run the computer program on the tangible medium or execute the instruction on a computer readable medium.

The CT data to be analyzed using the teachings of the present embodiments are generally in the form of imagery data arranged gridwise in a plurality of picture-elements (e.g., pixels, group of pixels, voxels, group of voxels, etc.).

The term “pixel” is sometimes abbreviated herein to indicate a picture-element. However, this is not intended to limit the meaning of the term “picture-element” which refers to a unit of the composition of an image.

References to an “image” or a “scan” herein are, inter alia, references to values at picture-elements treated collectively as an array. Thus, the terms “image” and “scan” as used herein also encompasses a mathematical object which does not necessarily correspond to a physical object. The original CT scans certainly do correspond to physical objects which are the body section from which the CT scans are acquired.

Each pixel in the scan is typically associated with a single digital intensity value, in which case the scan is represented as a grayscale image. Optionally, but not necessarily, each pixel can be associated with three or more digital intensity values sampling the amount of light at three or more different color channels (e.g., red, green and blue) in which case the scan is represented as a color image.

The CT data can conveniently be described as a matrix. Without loss of generality, a CT scan can be denoted as I(i, j, k), where I is a digital intensity value (or a set of digital intensity values in case of a color image), (i,j) denote in-plane coordinates of picture-elements, and k is a pointer to a plane containing the picture-element at (i, j). For example, k can be a slice number of the CT scan.

The method begins at 10 and optionally and preferably continues to 11 at which a pre-contrast administration CT scan of a body region is acquired. The body region is preferably extracranial region. Representative examples include, without limitation, the thorax, the abdomen, and the pelvis. In some embodiments of the present invention the body region is a lung of the subject, and in some embodiments of the present invention the body region includes two lungs of the subject.

The CT scan preferably encompasses at least a whole organ of the subject. For example, when the body region is the lung, the CT scan preferably encompasses the lung in its entirety, and when the body region includes two lungs the CT scan encompasses both lungs in their entirety.

The method optionally and preferably continues to 12 at which a detectable dose of a CT contrast agent is administered to the subject.

The contrast agent can be a positive contrast agent that provides a signal that is substantially greater than water (e.g., attenuation coefficient above 60 HU). These agents utilize materials with high X-ray attenuation properties. The contrast agent can alternatively be a neutral contrast agent that provides a signal that resembles that of water or soft tissue (e.g. attenuation coefficient between about −20 HU and about 60 HU). Still alternatively, the contrast agent can be a negative contrast agent that provides a signal that is substantially lower than that of water (e.g. attenuation coefficient below −20 HU).

As used herein the term “about” refers to ±10%.

Hydrocarbon oils, such as peanut oil or vegetable oil may be used as negative contrast agents. Commercial positive CT contrast agents are typically based on iodine or barium. The difference in x-ray attenuation between positive agents and the soft tissues of the body are nearly or wholly due to the iodine or barium since the other atoms in these agents typically resemble those of bodily soft tissue and water.

The term “detectable dose” refers to a dose which allows detection of the contrast agent in a CT X-Ray system. However, this need not necessarily be the case, since, for some embodiments, another type of contrast agent and/or another dose can be utilized.

The time at which the administration of contrast agent is initiated is denoted herein t=0. The method optionally and preferably continues to 13 at which a post-contrast administration CT scan is acquired at time t>0 as measured from t=0 (the initiation time of the contrast agent administration). The time t is referred to herein as a “delay time interval” representing a delay between the initiation of administration 12 of the contrast agent and the initiation of the acquisition time of the post-contrast administration scan. The “delay time interval” is preferably longer than the time required to administer the contrast agent, so that the post-contrast administration CT scan is acquired after the dose of contrast agent has been administrated in its entirety. Preferably, no CT scan is acquired during the administration of contrast agent. Typically, t is at least 60 seconds or at least 80 seconds or at least 120 seconds. This is unlike CTP in which the typical delay time is 1-5 seconds. Also contemplated are embodiments in which t is more than 120 seconds, e.g., more than 180 seconds, or more than 240 seconds, or more than 360 seconds, or more than 480 seconds, e.g., 500 e.g., seconds. Preferably, t is less than 1000 seconds.

The acquisition of each CT scan typically includes emission of an X-Ray beam at each of several projections and detection of the beam, once attenuated by the body, by a detector array to provide a set of CT slices forming the CT scan in which each CT slice corresponds to one of the projections. The number of CT slices in a set depends on the desired field of view and the slice thickness.

Each of the CT scans may be according to any scanning procedure, such as, but not limited to, contrast-enhanced CT scan, high dose, low dose or ultra low dose CT scan. The pre-contrast administration CT scan is a non-contrast scan. The scanner may be but is not limited to a spiral CT, Electron beam tomography, spectral CT, photon counting CT. Other scanners are also contemplated.

Operation 13 can include acquisition of a single post-contrast administration CT scan, or, optionally and preferably, acquisition of a sequence of post-contrast administration CT scans, at different times after the initiation time t=0 of the contrast agent administration. In these embodiments, there are several delay time intervals, one for each post-contrast administration CT scan of the sequence. These delay time intervals are denoted tₙ (n=1, 2, . . . ) with 0<t₁<t₂ etc. The differences between the acquisition times of sequence can all be the same or at least two such difference can be different, as desired. Thus, for example, when there are two post-contrast administration CT scans t₁ can be the same as t₂−t₁ or be different from t₂−t₁, when there are three post-contrast administration CT scans, t₁, t₂−t₁, and t₃−t₂ can all have the same values, or at least two of t₁, t₂−t₁, and t₃−t₂ can be different from each other, and so on. Preferably, for at least one, or at least two, or at least three, or each pair of successive post-contrast administration CT scans of the sequence, the time difference tₙ₊₁−tₙ between the acquisition times tₙ, tₙ₊₁ of the two scans is at least 30 seconds.

The CT scans (pre- and post-contrast administration) can be acquired according to the same scanning procedure, or according to the different scanning procedures. For example, in some embodiments both pre- and post-contrast administration CT scans are characterized by the same or approximately the same radiation dose, and in some embodiments the pre-contrast administration CT scan is characterized by a radiation dose that is different (e.g., higher or lower) than the post-contrast administration CT scan. For example, the post-contrast administration CT scan can be characterized by a radiation dose that is 5-10% of the radiation dose of the pre-contrast administration CT scan.

Alternatively, data describing the aforementioned CT scans can be obtained from an external source (e.g., read from a computer readable storage medium, or directly from the storage of the CT scanner, or downloaded over a communication network from a cloud storage facility, or a remote computer or CT scanner), in which case 11, 12 and 13 can be skipped.

In some embodiments of the present invention the method proceeds to 15 at which the data describing each of the CT scans are normalized. The normalization is typically with respect to a reference intensity value which remains substantially constant across the set of CT slices forming the respective scan. Such reference intensity value can be predetermined, or obtained, for example, from a phantom sample which can be scanned by CT together with the body region. During normalization, each intensity value of data is divided by the reference intensity value to provide a normalized intensity value.

Optionally, the method proceeds to 15 at which data registration is applied to the data describing each of the CT scans. The registration is optionally and preferably applied to compensate for motion artifacts between the scans, and also between slices of the same scan. The motion artifacts can include whole body movements, and/or motion of internal organs, such as the lungs during breathing or the heart during the cardiac cycle. The registration can include, for example, determining centroids within one or more of the slices of each scan and aligning the respective centroids of among the scans.

At 17, one or more subtraction maps are formed from the data. A subtraction map according to preferred embodiments of the present invention is a map of subtraction values obtained by subtracting data describing pre-contrast administration CT scan from data describing post-contrast administration CT scan, or, conversely, by subtracting data describing post-contrast administration CT scan from data describing pre-contrast administration CT scan.

Below, a subtraction map formed by subtracting data describing pre-contrast administration CT scan from data describing post-contrast administration CT scan, is referred to as a “forward subtraction map,” and a subtraction map formed by subtracting data describing post-contrast administration CT scan from data describing pre-contrast administration CT scan, is referred to as a “backward subtraction map.”

It is appreciated that backward subtraction map is equivalent to forward subtraction map up to an overall multiplication of all the subtraction values of the map by −1.

The subtraction map is preferably displayable in a manner that at least a few (e.g., at least three or at least four or at least five or at least six or at least seven) of the subtraction values are distinguishable from each other. For example, the subtraction map can be color-coded with different colors or different hues of a color correspond to different subtraction values, or grayscale-coded with gray levels corresponding to different subtraction values. The color or, equivalently, gray level, can vary over the map continuously or discretely.

The data describing the pre- and post-contrast administration CT scans are typically intensity values. Since regions of the CT scan that are occupied by the CT contrast are typically enhanced in terms of the intensity, the intensity of any particular region in the post-contrast administration CT scan is typically higher or the same as the intensity of this region in the pre-contrast administration CT scan. Thus, for a forward subtraction map, the minimal subtraction value is typically zero and all other values are positive, and for a negative subtraction map, the maximal subtraction value is typically zero and all other values are negative. In various exemplary embodiments of the invention one of the colors or gray levels of the map is allocated to represent a zero subtraction value, indicating no or substantially small change in the intensity between t=0 andt>0.

The subtraction values of the subtraction map are interchangeably referred to herein as the “signals” of the map. Thus, a reference to a “positive signal” or “negative signal” or “zero signal” with respect to a particular region over the map indicates that for that particular region the subtraction value is positive, negative or zero, respectively.

When there is a sequence of post-contrast administration CT scans, the method preferably forms at least two subtraction maps, corresponding to subtractions among different pairs of scans. In these embodiments, when it is desired to form forward subtraction maps, the pre-contrast administration CT scan data are subtracted from the nth post-contrast administration CT scan data, and when it is desired to form backward subtraction maps, the nth post-contrast administration CT scan data are subtracted from the pre-contrast administration CT scan data, so that each such subtraction forms one map.

Operation 16 is optionally and preferably performed slice-by-slice for the respective scans, so that each map is a sliced map including a set of map slices, where each map slice is formed by subtracting the respective slice of one of the scans from the respective slice of the other scan preferably after co-registration of the two scans. Thus, for example, denoting the CT data of the pre-contrast administration CT scan by I₀(i, j, k), and denoting the CT data of the nth post-contrast administration CT scan by Iₙ(i, j, k), a sliced forward subtraction map, containing k map slices, can be formed by performing the subtraction: Mₙ(i, j, k)=Iₙ(i, j, k)−I₀(i, j, k), and a sliced backward subtraction map, containing k map slices, can be formed by performing the subtraction: Mₙ(i, j, k)=I₀(i, j, k)−Iₙ(i, j, k).

Alternatively or additionally, operation 16 can be executed after averaging over the slices of each scan. In these embodiments, each scan is subjected to an averaging procedure to provide an intensity map and the subtraction is performed among the intensity maps. Such an averaging procedure is denoted mathematically as P(i,j)=<(I(i,j,k)>ₖ. An intensity map P corresponding to scan I thus includes a plurality of average values. For example, the average value P(i,j) corresponding to the in-plane coordinates (i, j) of the intensity map can be obtained by averaging over the intensities I(i,j,k=1), I(i,j,k=2), etc., namely the intensities as obtained from the in-plane coordinates (i, j) of the first slice of scan I, the in-plane coordinates (i, j) of the second slice of scan I, and so on.

Once the intensity maps are constructed from the respective scan data, the subtraction map can be obtained by subtracting one intensity map from the other. It is appreciated that when the subtraction map is formed by subtracting the intensity maps (containing average values), the subtraction map is an unsliced map, and therefore does not include the slice index k. Thus, for example, denoting the intensity map obtained from the pre-contrast administration CT scan by P₀(i, j), and denoting the intensity map obtained from the nth the post-contrast administration CT scan by Pₙ(i, j), a forward subtraction map can be formed by performing the subtraction: Mₙ(i, j)=Pₙ(i, j)−P₀(i, j), and a backward subtraction map can be formed by performing the subtraction: Mₙ(i, j)=P₀(i, j)−Pₙ(i, j).

The inventors found that the sliced and unsliced subtraction maps of the present embodiments identify leaking of contrast agent out of blood vessels, and are therefore useful in identifying and/or distinguishing various conditions of the subject, including, without limitation, microvasculature leakiness, vasculature leakiness (e.g., pulmonary vessel leakiness), inflammation (e.g., pulmonary inflammation), and fibrosis (e.g., pulmonary fibrosis).

Such a distinction is manifested by the values stored in the subtraction map. For example, when the subtraction map is a forward subtraction map, a negative or zero value is stored at coordinate (i,j) when the intensity value of the post-contract administration CT scan (or the average value of the intensity maps obtained this scan), at coordinate (i,j) is larger than or equal to the intensity value of the pre-contract administration CT scan (or the average value of the intensity maps obtained from this scan), at coordinate (i,j), and a positive value is stored at coordinate (i,j) when the intensity value of the post-contract administration CT scan (or the average value of the intensity maps obtained this scan), at coordinate (i,j) is lower than the intensity value of the pre-contract administration CT scan (or the average value of the intensity maps obtained from this scan), at coordinate (i,j).

Positive values (in a forward subtraction map) indicate accumulation of contrast agent, and therefore provide information regarding the amount of contrast that enters the location corresponding the respective coordinate. For example, positive values may be indicative of existence of a blood vessel or vascular mass (such as a tumor) at the respective locations. Zero or close to zero values indicate no accumulation of contrast agent, and therefore typically indicate normal or healthy tissue at the location corresponding the respective coordinate. Subtle positive values (less than the typical values for blood vessels, but more than the typical values for normal tissue) may indicate accumulation of contrast agent.

The Inventors unexpectedly found that such accumulation indicates existence of leaky blood vessels at the respective location. The high sensitivity to such vessel leakiness obtained by the maps may enable early detection or assist in distinguishing between various conditions, such as inflammation or fibrosis, cytotoxic tissue response to treatment or exposure to toxins or physical trauma, various diseases and more. The Inventors unexpectedly found that contrast accumulation can be identified even when the blood vessels are too small to be distinguished in the CT scan itself, making the map of the present embodiments useful also for identifying regions of microvasculature leakiness.

In backward subtraction maps, the above indicators are reversed, and so negative values may be indicative of existence of a tumor or blood vessel, zero or close to zero values still indicate normal or healthy tissue, and negative values (more than the typical values for blood vessels, but less than the typical values for normal tissue) indicate existence of leaky blood vessels.

The method optionally and preferably continues to 17 at which the subtraction map is displayed. In some embodiments of the present invention the map is displayed to show a sufficiently large portion of the scanned body region. For example, when the body region is the lung, and the map displays the lung in its entirety, and when the body region comprises two lungs, and the map displays both lungs in their entirety. This is unlike conventional subtraction maps (e.g., those employed in CTP) in which only a small region-of-interest is scanned and displayed.

According to preferred embodiments of the present invention, the subtraction map is displayed in a manner that identifies leaking of contrast agent out of blood vessels in the region. For example, the map can be displayed as color coded maps, in which the aforementioned subtle values (subtle positive values in forward subtraction maps, and subtle negative values backward subtraction map) are colored differently than other values.

In some embodiments of the present invention the displayed subtraction map is color coded with three or more distinct colors, wherein a first color represents subtraction values less than a first predetermined threshold, a second color represents subtraction values more than a second predetermined threshold which is higher than the first predetermined threshold, and a third color represents subtraction values between the two predetermined thresholds. Thus, for forward (or backward) subtraction maps, the first (resp. second) color identifies normal or healthy tissue, the second (resp. first) color identifies existence of a tumor or blood vessel, and the third color identifies existence of leaky blood vessels.

The colors can optionally and preferably also be shade-scaled, whereby different absolute values stored in the map are displayed using different shades of the respective color. For example, lower positive values can be displayed using lower shade of red (e.g., pink) and higher positive values can be displayed using higher shade of red (e.g., deep red).

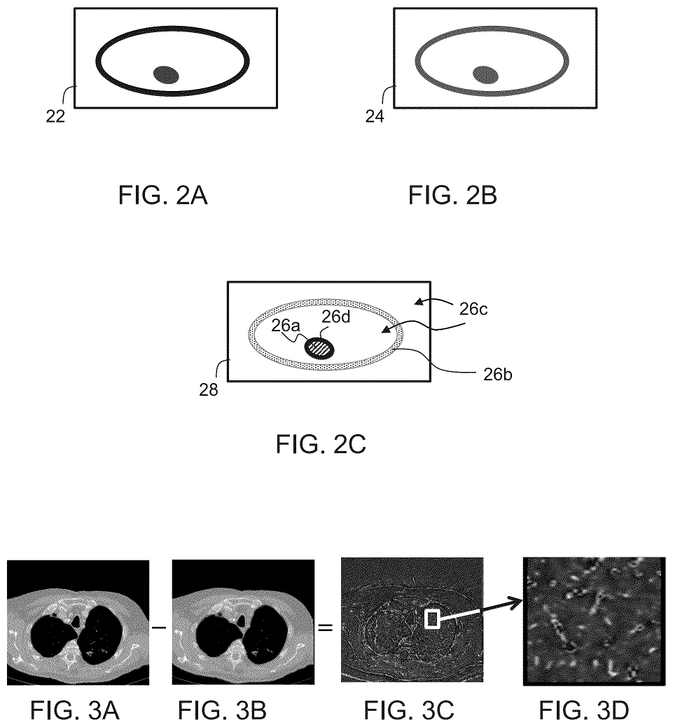

An exemplified distinction between normal tissue, tumor, blood vessel, and leaky blood vessel is illustrated in FIGS. 2A-C. FIG. 2A illustrates a post-contrast administration CT scan 22, and FIG. 2B illustrates a pre-contrast administration CT scan 24. Contours showing intensity enhancements due to X-ray attenuation differences between tissue regions containing the contrast agent and other tissue regions are depicted in the two scans. Due to the time difference at which the two scans 22 and 24 were acquired, the contrast of the shown contours is not the same between the two scan. However, within the scans, it is difficult to identify contrast differences, since the scans themselves do not depict variations as a function of the time.

FIG. 2C illustrates a forward subtraction map 28 formed by subtracting scan 24 from scan 22. Map 28 distinguish between different tissue regions. Two regions 26a and 26b in which the subtraction values are above the second (higher) threshold, where region 26a is more dense than region 26b, regions 26c in which the subtraction values are less than the first (lower) threshold, and a region 26d in which the subtraction values are between the two thresholds. Region 26a can be identified as a tumor, region 26b can be identified as a normal blood vessel, regions 26c can be identified as a normal tissue other than a blood vessel, and regions 26d can be identified as a leaky blood vessel.

FIGS. 3A-3D depict experimental results corresponding to the schematic illustrations of FIGS. 2A-2C, for the case of a body region that includes the lungs of a subject. In FIGS. 3A-3D, FIG. 3A is the post-contrast administration CT scan (acquired using a full dose standard scan) 22, FIG. 3B is the pre-contrast administration CT scan (acquired using a full dose standard scan) 24, and FIG. 3C is the forward subtraction map 28. The map 28 is color coded, showing regions in red, regions in blue, and regions in yellow. Regions in red correspond to a relatively large positive subtraction value (substantially higher intensity in the post-post-contrast administration CT scan relative to the pre-contrast administration CT scan), regions in yellow correspond to a medium positive subtraction value (but still higher intensity in the post-contrast administration CT scan relative to the pre-contrast administration CT scan), and blue regions correspond to a positive subtraction value which is zero or close to zero (no change in the intensity in the post-contrast administration CT scan relative to the pre-contrast administration CT scan). FIG. 3D shows a zoom-in region marked by a white rectangle on FIG. 2C. The regions in yellow and red (positive signal) correspond to blood vessels, and the regions in blue (no change in signal between the 2 scans) correspond to the background of the lungs which show no significant contrast leakage.

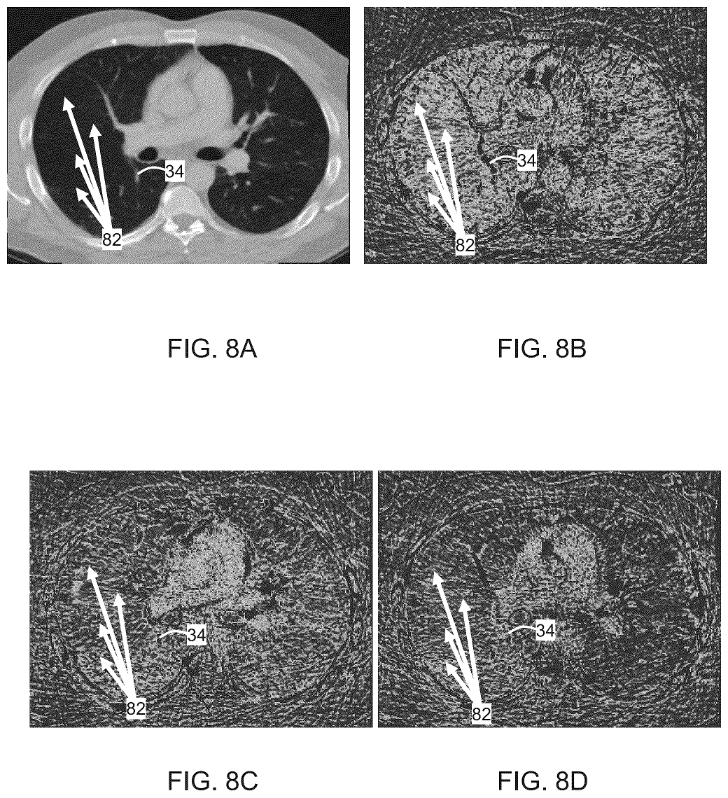

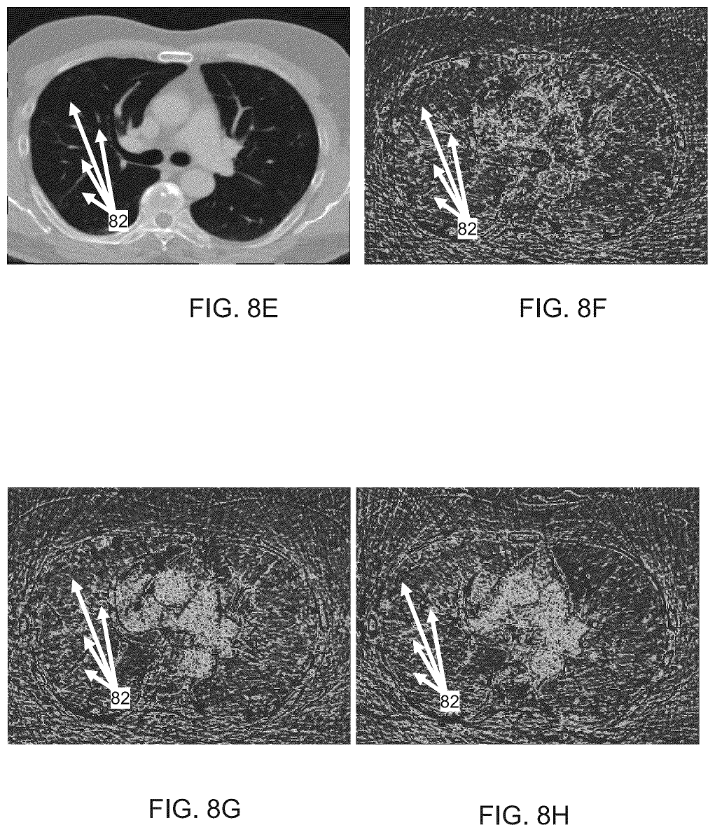

FIGS. 8A-8H show pre-contrast administration CT scans (FIGS. 8A and 8E) and subtraction maps (FIGS. 8B-8D and 8F-8H) of two patients recovering from Covid-19 virus yet still symptomatic, calculated by subtracting the pre-contrast full dose standard scan from 3 post-contrast ultra low dose CT scans (10% of the dose of a standard full dose scan), acquired at t=90 (FIGS. 8B and 8F), t=260 (FIGS. 8C and 8G) and t=501 (FIGS. 8D and 8H) seconds after initiation (t=0) of contrast injection. The right lung tissue is marked by arrows 82.

A first patient (FIGS. 8A-8D), was scanned 1.5 months after being hospitalized due to COVID19. The map of FIG. 8B (corresponding to t=90 seconds) shows substantially large regions of lung tissue with positive signals (color-coded as yellow and red pixels), indicating a significant vessel leakiness. A representative example of a pulmonary blood vessel is shown at 34. The leakiness (e.g., of vessel 34) is depicted mainly in the map calculated 90 seconds post contrast (FIG. 8B), where in the later maps (FIGS. 8C-D), most of the contrast is cleared from the lungs.

A second patient (FIGS. 8E-8H), was scanned 5 months after being hospitalized due to COVID19. Unlike the first patient, the signal of all three maps for this patient is small or zero (color-coded as blue pixels) for most of the lung tissue, indicating no significant vessel leakiness for this patient, similarly to the patients of FIGS. 3A, 4B, 5A-C.

The maps of the present embodiments are therefore useful in differentiating symptoms arising from an active inflammation (e.g., virus-induced inflammation), from those arising from non-inflammatory damage (for example fibrotic damage after the inflammation has ceased), wherein when vessel leakiness is identified, the method can determine that the tissue region has an active inflammation, and when no, or substantially no, vessel leakiness is identified, the method can determine that the tissue region has a non-inflammatory damage.

The present embodiments are useful in early detection of disease (e.g., viral, bacterial, vascular). For example, since viruses in the lungs often induce vessel leakiness as part of their mechanism of action, being highly sensitive to vessel leakiness may enable early detection of the disease and early characterization of how it will evolve.

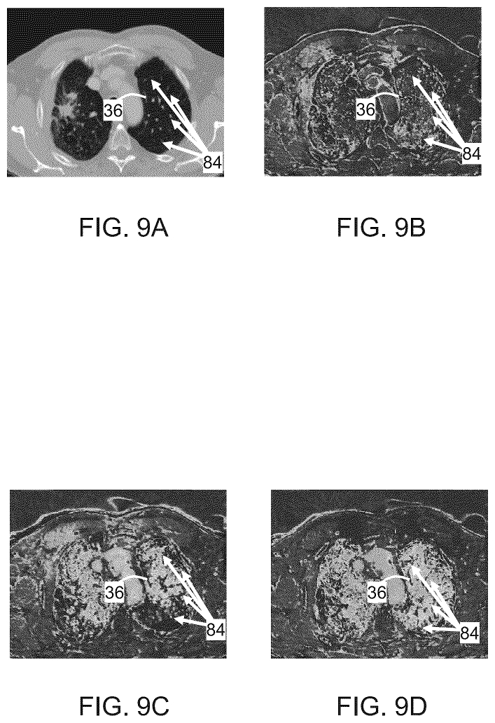

The present embodiments can also be in detection of cytotoxicity caused by a treatment (for example treatment for cancer), or by exposure (e.g., by breathing toxic gas), or trauma. FIGS. 9A-9D show post-contrast administration CT scan (FIG. 9A) and subtraction maps (FIGS. 9B-9D) of a patient with lung cancer during immune therapy, calculated by subtracting the pre-contrast scan from 3 post-contrast CT scans acquired at t=80 (FIG. 9B), t=200 (FIG. 9C) and t=380 (FIG. 9D) seconds after initiation (t=0) of contrast injection. The left lung tissue is marked by arrows 84, and a representative example of pulmonary blood vessel is shown at 36. The maps of this patient shows substantially large regions of lung tissue with positive signals (color-coded as yellow and red pixels), for both the left and right lungs, with a peak at FIG. 9C (corresponding to t=200 seconds), indicating a leakiness of many blood vessels, including the pulmonary blood vessel at 36.

The present embodiments can also be useful for early detection of COVID19 in patients which are not symptomatic yet and predicting deterioration.

The present embodiments can be useful in early detection of changes (for example, progression or recovery of a disease or cytotoxicity, and can also provide prediction of such progression or recovery.

The present embodiments can be useful for disease staging and/or characterization. For example, some types of diseases may show one pattern of vessel leakiness while another disease shows a different pattern, and so by analyzing the pattern of vessel leakiness, the disease can be characterized or staged. For example, in FIGS. 8A-D (patient recovering from COVID19) maximal contrast accumulation is observed in the lungs 90 seconds post contrast injection (FIG. 8B), while in FIGS. 9A-D (patient with lung cancer during immune therapy) maximal contrast accumulation is observed in the lungs 200 seconds after contrast injection.

The analysis can include a comparison to a library of previously identified patterns of vessel leakiness and their association to specific diseases or disease stages.

The present embodiments can be useful for differentiating inflammation from non-inflammatory pathologies such as fibrosis. For example, when vessel leakiness is identified, the method can determine that the tissue region includes inflammation, and when no or reduced level of vessel leakiness is identified, the method can determine that the tissue region includes a non-inflammatory pathology.

The present embodiments can be useful for predicting transplant rejection, such as, but not limited to, rejection of transplanted lungs. This is advantageous because the likelihood of transplant rejection during the first year following lung transplantation is considerable (about 30%). Rejection of transplanted lungs is typically accompanied by inflammation, and the Inventors found that such a rejection increases vessel leakiness which can be identified according to some embodiments of the present invention by means of the aforementioned subtraction maps.

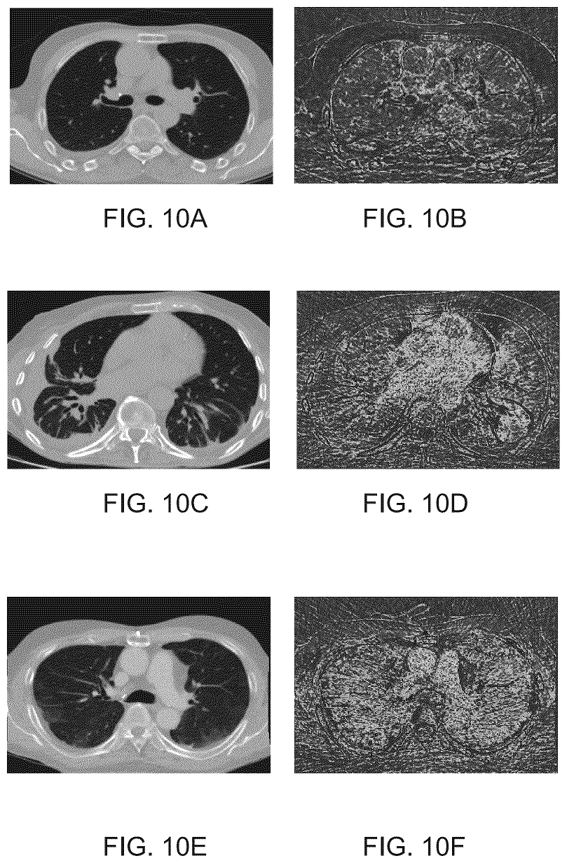

In experiments performed by the Inventors, 12 patients that underwent lung transplantation, and for which biopsy was extracted regularly (every three months), were scanned pre- and post-contrast administration and subtraction maps were formed in accordance with embodiments of the present invention. Of these 12 patients, 10 patients did not reject the transplanted lungs, and 2 patients rejected the transplanted lungs, as confirmed by the biopsy. FIG. 10A shows a pre-contrast administration CT scan of one of the patients that did not reject the transplanted lungs, and FIG. 10B shows the corresponding subtraction map for this patient. As shown, the signal of the map at FIG. 10B is small or zero (color-coded as blue pixels) for most of the lung tissue, indicating no significant vessel leakiness for this patient. FIGS. 10C and 10E shows pre-contrast administration CT scans of the two patients that reject the transplanted lungs, and FIGS. 10D and 10F show, respectively, the corresponding subtraction map for these patients. Unlike the map in FIG. 10B, the maps of FIGS. 10D and 10F show substantially large regions of lung tissue with positive signals (color-coded as yellow and red pixels), indicating a significant vessel leakiness.

Following the display of the maps, and optionally and preferably also the identification or prediction of the condition of the tissue or the subject, the method can proceed to 20 at which it ends.

Alternatively, the method can proceed to 18 at which treatment is applied to the subject, responsively to one or more of the types of tissue that are identified by the map. The treatment can include treatment by chemical or biological medication, by radiation, or invasive or partially-invasive treatment.

For example, when the subtraction map identifies a region of vessel leakiness (e.g., pulmonary vessel leakiness), the applied treatment is directed to treat vessel leakiness (for example, using angiopoietin-2 antagonist compound or a compound having similar function), when the subtraction map identifies a region of inflammation (e.g., pulmonary inflammation), the applied treatment is directed to treat inflammation (for example, using a steroid or an A_{2B} receptor antagonist or a nucleotidase or a compound having similar function), and when the subtraction map identifies a region of fibrosis (e.g., pulmonary fibrosis), the applied treatment is directed to treat fibrosis (for example, using an inhibitor of the activity of one more proteins for which increment in activity is associated with fibrosis). Also contemplated are inhaled treatments, such as anti-inflammation agents, e.g., steroids when lung inflammation is identified. Furthermore, the subtraction map of the present embodiments can also identify different regions in the lungs with different conditions, and treatments that are targeted to different problems can be delivered to the respective region, e.g., by bronchoscopy, using specific drugs, or other interventions.

The method ends at 20.

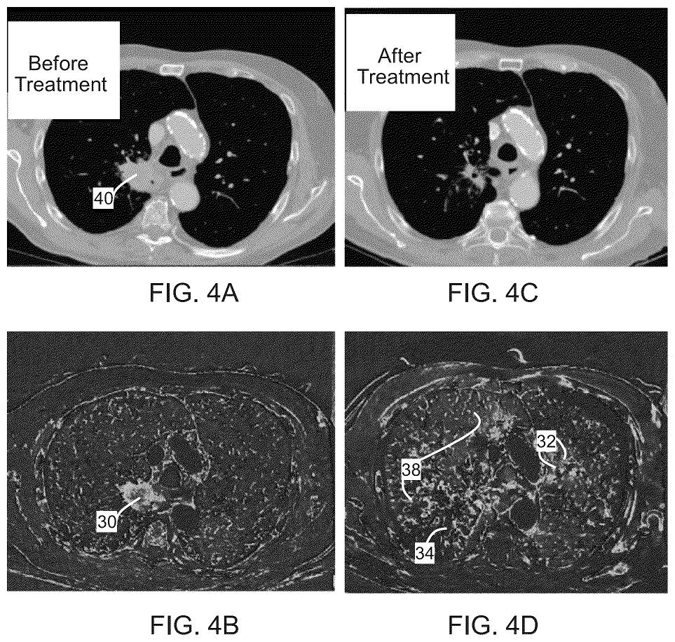

The method of the present embodiments can be used for assessing the effect of treatment. A representative example of such embodiments is shown in FIGS. 4A-D, which show the cytotoxic effects of the antineoplastic treatment on the lungs. FIGS. 4A and 4C are post-contrast administration CT scans of acquired from the lungs of a subject with lung cancer, 80 seconds after contrast administration, and FIGS. 4B and 4D are the respective forward subtraction maps, before treatment (FIGS. 4A-B) and 2 months post treatment by chemo-radiotherapy (FIGS. 4C-D). The lesion before treatment is shown at 30. The maps (FIGS. 4B and 4D) are colored using the same color codes as described above with respect to FIG. 3C. Before treatment (FIG. 4B), the lungs appear blue, indicating that there is no vessel leakiness. After treatment (FIG. 4D), a moderate accumulation of contrast is observed, mainly in the lung appearing on the left side of the map (shown at 38), indicating leakiness of the vessels the lung's area, and to a lesser extent in the contra-lateral lung (shown at 32), indicating leakiness of microvasculature. It can also be seen that the vessels in the right lung in the map, are significantly enlarged, suggesting vessel leakiness also of larger pulmonary vessels (shown at 34). FIGS. 4B and 4D thus demonstrate that the method of the present embodiments allows assessing the effect of a treatment (higher leakiness after chemo-radiotherapy, in this example).

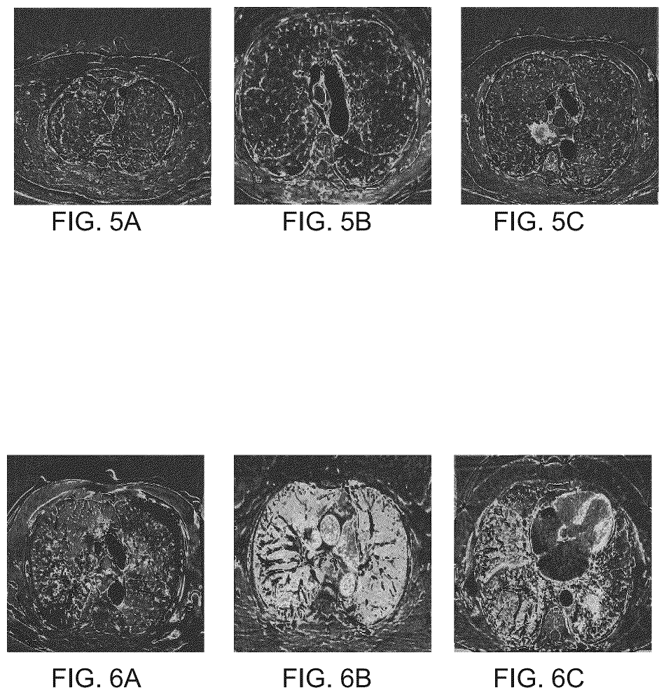

FIGS. 5A, 5B and 5C are forward subtraction maps obtained, respectively, for three subjects with lung cancer prior to treatment, showing no significant vessel leakiness. The forward subtraction maps FIGS. 5A, 5B and 5C were obtained by subtracting a full dose pre-contrast administration CT scan from a full dose post-contrast administration CT scan acquired seconds after contrast administration.

FIGS. 6A, 6B and 6C are forward subtraction maps obtained, respectively, for the three subjects with lung cancer after or during treatment, showing various degrees of vessel leakiness. The forward subtraction maps FIGS. 6A, 6B and 6C were obtained by subtracting a full dose pre-contrast administration CT scan from a full dose post-contrast administration CT scan acquired seconds (FIG. 6A), 90 seconds (FIG. 6B), and 80 seconds (FIG. 6C), after contrast administration.

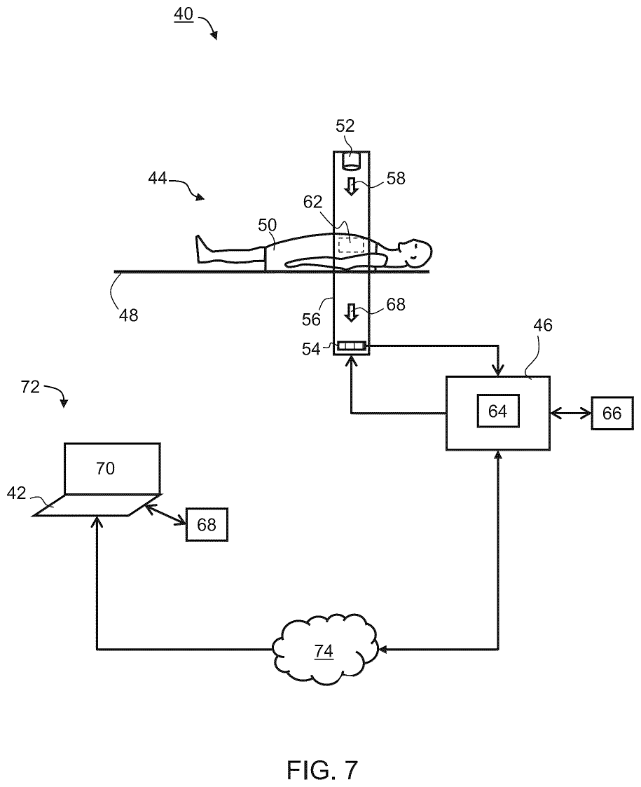

FIG. 7 is a schematic illustration of a CT system 40, according to some embodiments of the present invention. CT system 40 comprises an image processor 42, a CT scanner 44 and a computerized controller 46.

CT scanner 44 comprises a bed 48 for supporting a subject 50, an x-ray source 52 producing a collimated x-ray beam 58, and a detector array 54, configured for detecting an attenuated x-ray beam 60 formed by the interaction of beam 58 with a body region 62 of subject and responsively generating an electrical signal, such as a video signal. Source 52 and detector 54 are mounted on an annular gantry 56 at opposite sides of the bed 48. Gantry 56 controls the position of detector 54 and source 52, and is configured to rotate around bed 48 together with detector 54 and source 52, so as to scan the direction of ray beam 58.

It is expected that during the life of a patent maturing from this application many relevant CT scanners will be developed and the scope of the term CT scanners is intended to include all such new technologies a priori.

CT scanner 44 is controlled by computerized controller 46. The signal provided by detector 54 at each projection of gantry 56 is communicated to controller 46. Computerized controller 46 has image capture hardware 64, configured to collect the signal for each projection, to digitize the signals, and to calculate from the digitized signals a tomogram of the body region 62, thereby providing a CT scan. In some embodiments of the present invention controller 46 is associated with a memory medium 66, preferably a non-transitory medium, which stores computer programs for calculating the tomogram, and which may also store the slices of CT scan, once generated, e.g., in the form of a plurality of images. Controller 46 also controls the angular position of gantry 48, and the operation of source 52. In various exemplary embodiments of the invention controller 46 controls CT scanner 44 and acquires the pre-contrast administration CT scan and the post-contrast administration CT scan(s), as further detailed hereinabove.

Processor 42 can be local or remote with respect to scanner 44. The I/O circuits (not shown) of controller 46 and processor 42 can communicate information with each other via a wired or wireless communication. For example, controller 46 and processor 42 can communicate via a network 74, such as a direct cable, local area network (LAN), a wide area network (WAN) or the Internet. In some embodiments of the present invention processor 42 is a component in a server computer 72 that is remote from CT scanner 44. For example, server computer 72 can in some embodiments be a part of a cloud computing resource of a cloud computing facility in communication with controller 46 over network 74.

Data describing the CT scans are communicated from controller 46 to image processor 42. Processor 42 is associated with a computer readable storage medium 68 tangibly embodying a program of instructions executable by processor 42 to receive the data, to form one or more subtraction map from the data by subtracting one of the scans from another as further detailed hereinabove. In some embodiments of the present invention Processor 42 stores data describing the map in a computer readable storage, e.g., storage 68. Processor 42 preferably displays the subtraction map on a display 70 in a manner that identifies leaking of contrast agent out of blood vessels in the region, as further detailed hereinabove. Display 70 can be located locally with respect to processor 42, as shown in FIG. 7, or at a location that is remote from processor 42 (e.g., near scanner 44, at an oncologist's clinic, etc.). Also contemplated, are embodiments in which display 70 is a GUI of a mobile device such as a smartphone, a tablet, a smartwatch and the like. When display 70 is remote from processor 42 or a display of a mobile device, data describing the subtraction map is transmitted over network 74 to display 70.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method of analyzing CT data describing a body region of a subject administered with contrast agent, the method comprising:
receiving data describing a pre-contrast administration CT scan from a body region of the subject and a post-contrast administration CT scan acquired from said body region at least 1 minute after initiation of administration of contrast agent to the subject;
forming a subtraction map from said data by subtracting one of said scans from another; and
displaying said subtraction map in a manner that identifies leaking of contrast agent out of blood vessels in said region.

2. The method of claim 1, comprising acquiring said pre-contrast administration CT scan, administering said contrast agent to the subject, and acquiring said post-contrast administration CT scan.

3. The method of claim 2, wherein said acquiring is by a CT scanner, and the method comprises idling said CT scanner between said pre- and said post-contrast administration CT scans.

4. The method according to claim 1, wherein said post-contrast administration CT scan is acquired t seconds after said contrast administration, said t being at least 80 seconds.

5. The method according to claim 4, wherein said t is at least 120 seconds.

6. The method according to claim 4, wherein said t is at least 240 seconds.

7. The method according to claim 4, wherein said t is at least 360 seconds.

8. The method according to claim 1, wherein there is a plurality of post-contrast administration CT scans, and the method comprises forming a subtraction map for each of said plurality of post-contrast administration CT scans, relative to said pre-contrast administration CT scan, thereby forming a plurality of subtraction maps.

9. The method according to claim 8, wherein for each subtraction map, a time delay between acquisition times of said pre-contrast administration CT scan and a respective post-contrast administration CT scan is at least 1 minute.

10. The method according to claim 1, wherein said displaying said subtraction map comprises identifying on said map a region of microvasculature leakiness.

11. The method according to claim 1, wherein said body region comprises two lungs, and said map displays both said lungs in their entirety.

12. The method according to claim 1, wherein said displaying said subtraction map comprises identifying on said map a region of pulmonary vessel leakiness.

13. The method according to claim 12, comprising treating the subject for said pulmonary vessel leakiness.

14. The method according to claim 1, wherein said displaying said subtraction map comprises identifying on said map a region of inflammation.

15. The method according to claim 14, comprising treating the subject for said inflammation.

16. The method according to claim 1, wherein said displaying said subtraction map comprises identifying on said map a region of pulmonary fibrosis.

17. The method according to claim 16, comprising treating the subject for said pulmonary fibrosis.

18. The method according to claim 1, wherein said displayed subtraction map is color coded with at least three distinct colors, wherein a first color represents subtraction values less than a first predetermined threshold, a second color represents subtraction values more than a second predetermined threshold which is higher than said first predetermined threshold, and a third color represents subtraction values between said predetermined thresholds.

19. A computer software product, comprising a computer-readable medium in which program instructions are stored, which instructions, when read by a data processor, cause the data processor to receive data describing a pre-contrast administration CT scan from a body region of the subject and a post-contrast administration CT scan acquired from said body region at least 1 minute after administration of contrast agent to the subject, to form a subtraction map from said data by subtracting one of said scans from another, and to display said subtraction map in a manner that identifies leaking of contrast agent out of blood vessels in said region.

20. A CT system, comprising:
a CT scanner configured for acquiring CT scans;
a controller configured for controlling said CT scanner to acquire a pre-contrast administration CT scan from a body region of the subject, idle said CT scanner for at least 1 minute, and acquire a post-contrast administration CT scan from said body region; and
an image processor configured to receive data describing said pre- and post-contrast administration CT scans, to form a subtraction map from said data by subtracting one of said scans from another, and to display said subtraction map in a manner that identifies leaking of contrast agent out of blood vessels in said region.
